(19) United States
(12) Patent Application Publication
(10) Pub. No.: US 2025/0110118 A1
(43) Pub. Date: Apr. 3, 2025

(54) GLYCAN PEAK ASSIGNMENT

(71) Applicant: AGILENT TECHNOLOGIES, INC., Santa clara, CA (US)

(72) Inventors: Eric LYSTER, Santa Clara, CA (US); Oscar Potter, Santa Clara, CA (US); Randall Robinson, Santa Clara, CA (US)

(73) Assignee: AGILENT TECHNOLOGIES, INC., Santa Clara, CA (US)

(21) Appl. No.: 18/650,999

(22) Filed: Apr. 30, 2024

Related U.S. Application Data
(60) Provisional application No. 63/587,593, filed on Oct. 3, 2023.

(51) Int. Cl. G01N 33/53; G01N 30/72; G01N 30/86; G16B 40/10

(52) U.S. Cl. CPC G01N 33/5308; G01N 30/7233; G01N 30/8631; G16B 40/10

(57) ABSTRACT

In some examples, an apparatus may include a retention time (RT) receiver that is executed by at least one hardware processor, to receive glucose ladder retention times (RTs), and receive neutral and charged glycan RTs. An RT analyzer that is executed by the at least one hardware processor may determine, based on application of the glucose ladder RTs, and the neutral and charged glycan RTs to expected glucose unit (GU) values, adjusted GU values. Further, an expected RT generator that is executed by the at least one hardware processor may determine, based on the adjusted GU values, expected RTs for specified glycans.

### RELATED APPLICATION

This application claims priority to co-pending U.S. Provisional Patent Application Ser. No. 63/587,593, filed Oct. 3, 2023, titled “GLYCAN PEAK ASSIGNMENT”, the disclosure of which is hereby incorporated by reference in its entirety.

### BACKGROUND

With respect to glycans, releasing glycans from a glycoprotein and labelling the glycans with a fluorescent tag facilitates relative quantitation of each different glycan structure in a sample. In one example, liquid chromatography (LC) may be utilized to separate the labelled released glycans.

### BRIEF DESCRIPTION OF DRAWINGS

Features of the present disclosure are illustrated by way of example and not limited in the following figure(s), in which like numerals indicate like elements, in which:

FIG. 1 illustrates a layout of a glycan peak assignment apparatus in accordance with an example of the present disclosure;

FIG. 2 illustrates a flow chart of utilization and determination of observed glucose units (GU), expected GU, and predicted GU to illustrate operation of the glycan peak assignment apparatus of FIG. 1, in accordance with an example of the present disclosure;

FIG. 3 illustrates a flow chart of a scaling process to illustrate operation of the glycan peak assignment apparatus of FIG. 1, in accordance with an example of the present disclosure;

FIG. 4 illustrates a flow chart and an associated example of generation of hypothetical retention times (RTs) for degree of polymerizations (d.p.s) beyond those observed experimentally by fitting to a function to illustrate operation of the glycan peak assignment apparatus of FIG. 1, in accordance with an example of the present disclosure;

FIG. 5 illustrates improvement in RT prediction accuracy upon implementing an adjustment value to illustrate operation of the glycan peak assignment apparatus of FIG. 1, in accordance with an example of the present disclosure;

FIG. 6 illustrates an example block diagram for glycan peak assignment in accordance with an example of the present disclosure;

FIG. 7 illustrates a flowchart of an example method for glycan peak assignment in accordance with an example of the present disclosure; and

FIG. 8 illustrates a further example block diagram for glycan peak assignment in accordance with another example of the present disclosure.

### DETAILED DESCRIPTION

For simplicity and illustrative purposes, the present disclosure is described by referring mainly to examples. In the following description, numerous specific details are set forth in order to provide a thorough understanding of the present disclosure. It will be readily apparent however, that the present disclosure may be practiced without limitation to these specific details. In other instances, some methods and structures have not been described in detail so as not to unnecessarily obscure the present disclosure.

Throughout the present disclosure, the terms “a” and “an” are intended to denote at least one of a particular element. As used herein, the term “includes” means includes but not limited to, the term “including” means including but not limited to. The term “based on” means based at least in part on.

Glycan peak assignment apparatuses, methods for glycan peak assignment, and non-transitory computer readable media having stored thereon machine readable instructions to provide glycan peak assignment are disclosed herein.

With respect to release of glycans from a glycoprotein and labelling of the glycans with a fluorescent tag, it can be technically challenging to assign a peak to a glycan structure. In some cases, the retention time (RT) of peaks may be utilized to determine the identity of a glycan structure. However, utilization of the retention times may include drawbacks in that the retention times may vary significantly from day to day, over the life of a chromatographic column, from poorly controlled parameters such as ambient temperature effects, due to small changes in the composition of the liquid chromatography (LC) mobile phase, and/or between individual chromatography columns of the same type.

The selectivity between different oligosaccharides (a) may be relatively stable for separation modes associated with hydrophilic interaction chromatography (HILIC), versus their relatively unstable absolute RTs. In this regard, a carbohydrate standard may be utilized to normalize the retention times, creating a retention index (RI), which may be used to control the aforementioned variations. The RI for a glycan structure may be more stable than the retention time, and may therefore be more suitable for assigning peaks to structures. In one example, peaks may be assigned to glycan structures by referring to a library of expected RIs developed based on separate experimental results with previously encountered glycans with well-confirmed structures.

In one example, implementation of an RI may rely on running a glucose homopolymer mixture (e.g., “ladder”), such as dextran, which is labeled with the same tag as the sample (such as 2-aminobenzamide, or InstantPC). Examples of standards such as the 2-aminobenzamide (2-AB) labelled glucose homopolymer standard, InstantPC labelled maltodextrin standard, etc., may be run as part of the same analysis sequence (e.g., a consecutive run before or after the sample runs) and utilize the same instrument method as possible, including mobile phase composition gradient, flow rate and column temperature. The results from a calibration run may be used to fit the relationship between RT and degree of polymerization of the glucose homopolymer with a function such as a 5^{th} degree polynomial function. This approach may be utilized for both size exclusion chromatography mode and HILIC mode chromatography of glycans. For each sample run, the same function may be used to map the RT of each glycan peak to a glucose units (GU) number that represents the degree of polymerization of a labeled glucose homopolymer that would elute at the same time as the glycan peak in question. In some cases, these may be a hypothetical fractional degree of polymerization such as 5.43, 7.81 etc. (e.g., fractional GUs that fall between the RT of two peaks in the labelled glucose homopolymer standard).

Implementing an RI approach may help prevent uncertainty over peak identity in an LC run if an experimenter is performing routine analysis and sees changes in the RT, since the experimenter is aware that the shift is due to changes in their system, as opposed to a new glycan structure appearing in the sample.

It is also possible to assign unknown glycan structures by referring to a list of previously confirmed glycan structures (e.g., labeled with the same tag) that were previously shown to give various GU numbers under very similar experimental conditions. Such lists of glycan structures and their GU may be referred to as a GU library or GU database, such as the library known as Glycobase. In some HILIC methods, glycan structures on amide HILIC columns with common tags may elute in the range from 4 to 14 GU, although this range can be higher or lower depending on the column and technique utilized.

The same general principles may be used with electrophoresis methods, in which case the labeled homopolymer standard migration times may be used to create a migration index that achieves the same goals as the retention index. Tables of known structures and their corresponding GU values may be utilized for assigning glycan structures with neutral tags such as 2-aminobenzamide, but may be less accurate and therefore less useful when working with positively charged tags such as InstantPC. This is because of the higher charge heterogeneity of such samples. For example, when glycan samples containing structures with acidic groups are labeled with the positively charged tag, they may have a net 0 or negative charge, unlike the uniformly positively charged homopolymer mixture. In such cases, the GU values may be more sensitive to changes in ionic interactions brought about by experimental conditions that cannot readily be controlled, such as individual column characteristics, column age, and precise mobile composition. Regardless of the cause, the approach to assigning unknown peaks based on their GU values may be less reliable with positively charged tags.

In order to address at least the aforementioned technical challenges, the apparatuses, methods, and non-transitory computer readable media disclosed herein apply an adjustment to the expected GU (or RT) of each glycan structure in a library according to the number of acidic monosaccharides it contains, which can be 0, 1, 2, 3 or 4. These “charge groups” of glycans are known, respectively, as S0, S1, S2, S3 and S4. The magnitude of the adjustment applied to the GU (or expected RT) may be determined using the RT of labeled glycans of known structure, either as adjacent, external RT standards, or internal RT standards if some peaks in the sample itself have been confidently assigned. For example, the observed GU of an S0 glycan such as GOF may be determined and then the deviation from the expected value (from a “library” of expected GU values) may be applied as an adjustment to the predicted GU/RT of all other S0 glycans.

With respect to S2 glycans, the observed deviation of GU/RT of an observed S2 glycan peak of a known structure may be determined and applied as an adjustment to the predicted GU/RT of all other S2 glycans. This may be done for all charge groups, or even other types of structural glycan categories such as high mannose glycans.

However, to limit the burden of obtaining many calibration points, effective adjustment values for S1, S3 and S4 glycans may be determined by scaling up or down the adjustments from the S0 or S2 glycans.

The apparatuses, methods, and non-transitory computer readable media disclosed herein thus provide for a user to enter observed RTs for their glucose homopolymer standard and enter RTs for an S0 and an S2 glycan. A library of GU values may be provided for labelled glycans that have been determined experimentally using a HILIC column and separation method. When the apparatus is operated after submittal of calibration retention times by a user, the apparatus may determine the predicted RTs at which the user should expect to see each glycan in a library on their own system.

For the apparatuses, methods, and non-transitory computer readable media disclosed herein, in one example, the resultant library of expected RTs may be automatically (e.g., without human intervention) formatted as a compound database or. CSV file, and imported into a liquid chromatography (LC)/mass spectrometry (MS) data analysis software package such as Masshunter Bioconfim. Using this database, the software package may automatically assign the signals observed in LC/MS sample data to glycan structures based on both the RT and their mass to charge ratios as detected by a mass spectrometer, providing a relatively higher degree of confidence in the peak assignment compared to the approach of using RT or mass to charge ratio alone. For example, the calculated expected RTs, as disclosed herein, may be exported as a .CSV file that contains, in the following order of comma delimited columns: the chemical formulae of each glycan structure, the expected RTs of each glycan in the library, a blank column, the name of the glycan structure, and a final column for comments. This .CSV file may be loaded as a compound database file, for example, in the data analysis software package. The data analysis software package may be utilized for automatic searching through LC/MS data for signals that match the expected masses of compounds in a compound database. A table of compounds detected along with a certainty score may be output, and the certainty score may be based on how closely the observed mass for a given compound fits the expected mass based on the chemical formula. This allows a user to quickly identify compounds for signals observed in their LC/MS data. The data analysis software package may include additional features that can be used when the compound database includes accurate expected RTs. In that case, the certainty score may be based on the observed versus expected RT of the proposed compounds. In the case of MassHunter Bioconfirm, the RT certainty score may be a linear function where 0 deviation gives the maximum score of 100. The score drops off linearly down to zero when the deviation from the expected RT reaches a certain value, which can be set by a user. The ability to easily score accuracy or compare the RTs to their expected values is especially useful for glycans with more than one isomer, which cannot be distinguished by mass but may instead be distinguished by RT. The output table may also display the observed versus expected RTs for each signal so that a user can see if the deviation is low or large enough to arouse suspicion that the proposed compound is incorrect. Thus the highly accurate RT predictions achieved by the apparatuses, methods, and non-transitory computer readable media disclosed herein greatly enhances the accuracy and certainty achieved when using a data analysis software package to automatically analyze LC/MS data from glycan samples.

The apparatuses, methods, and non-transitory computer readable media disclosed herein provide technical benefits such as an air gap that separates a user's personal data from the software platform of the apparatus. A user may likely collect both confidential and non-confidential experimental data, but the user is only required to enter non-confidential data into the software platform. This data is then processed by the software platform and returned to the user as a list of predicted retention times, which assists the user in interpreting their data. Furthermore, the ability of the apparatuses, methods, and non-transitory computer readable media disclosed herein to provide predicted retention times eliminates the need to understand how to compare GU values to assign peak identities. In one example disclosed herein, the ability to predict retention times is aided by the use of a novel polynomial that expresses retention time as a function of GU. This polynomial may be paired with a novel extrapolation feature as disclosed herein. The implementation of glycan adjustment values as disclosed herein provides another technical advancement that improves the accuracy of retention time predictions and allows calibration refinement using internal sample peaks.

The apparatuses, methods, and non-transitory computer readable media disclosed herein generally include first generating GU values using a glucose homopolymer standard and then adjusting with real glycan RTs. In this regard, the apparatuses, methods, and non-transitory computer readable media disclosed herein may be implemented based on glycan RTs.

According to another aspect of the apparatuses, methods, and non-transitory computer readable media disclosed herein, some glycans elute at a retention time that is greater than that of the highest degree of polymerization peaks that can be reliably integrated using available standards of labelled glucose homopolymer. This presents technical challenges when interpolating from a 5^{th} degree polynomial that is valid from the lowest degree of polymerization until the highest degree of polymerization that was used to fit the function. In this regard, hypothetical RTs may be generated for degree of polymerizations beyond those observed experimentally by fitting to a function, such as a linear function, based on the final 2 or 3 points (e.g., the RTs of the highest, second highest and third highest degree of polymerization labelled glucose homopolymer peaks). These hypothetical glucose homopolymer degree of polymerization retention times may be treated as though they were experimentally observed, allowing the 5^{th} degree polynomial to extend to higher degree of polymerization (and thus higher GU/RT) than would otherwise be possible.

For the apparatuses, methods, and non-transitory computer readable media disclosed herein, the elements of the apparatuses, methods, and non-transitory computer readable media disclosed herein may be any combination of hardware and programming to implement the functionalities of the respective elements. In some examples described herein, the combinations of hardware and programming may be implemented in a number of different ways. For example, the programming for the elements may be processor executable instructions stored on a non-transitory machine-readable storage medium and the hardware for the elements may include a processing resource to execute those instructions. In these examples, a computing device implementing such elements may include the machine-readable storage medium storing the instructions and the processing resource to execute the instructions, or the machine-readable storage medium may be separately stored and accessible by the computing device and the processing resource. In some examples, some elements may be implemented in circuitry.

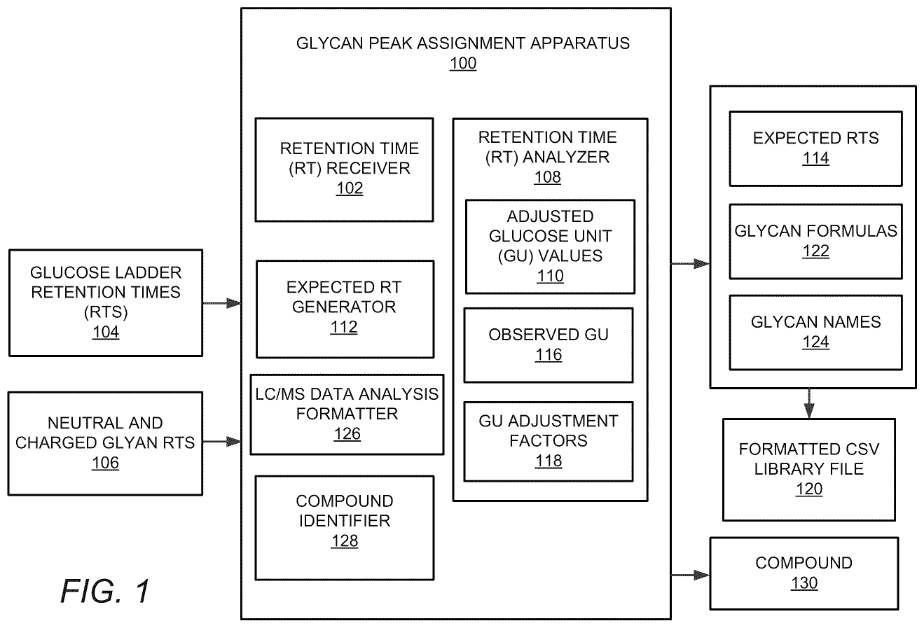

FIG. 1 illustrates a layout of an example glycan peak assignment apparatus (hereinafter also referred to as “apparatus 100”).

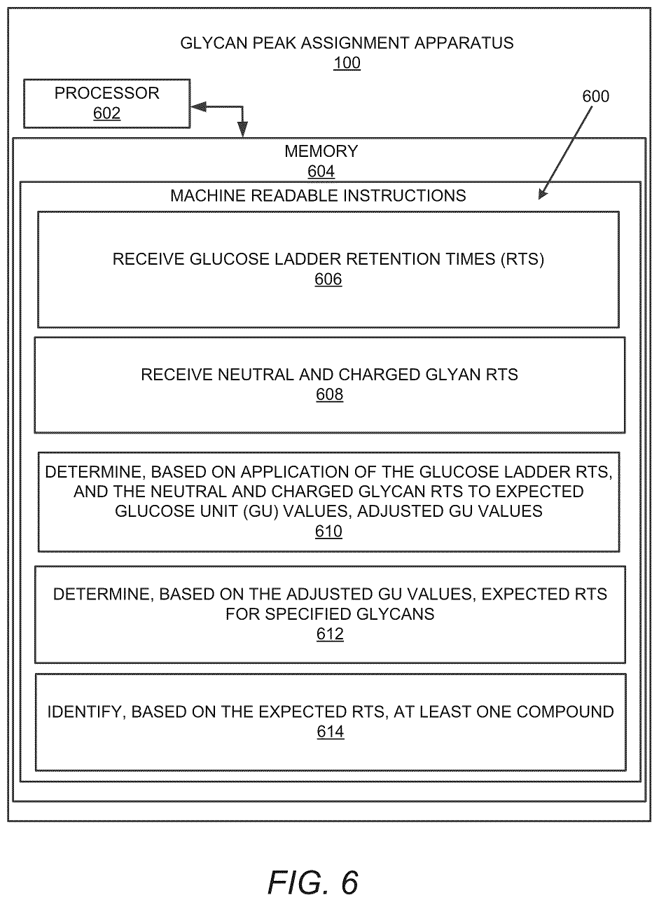

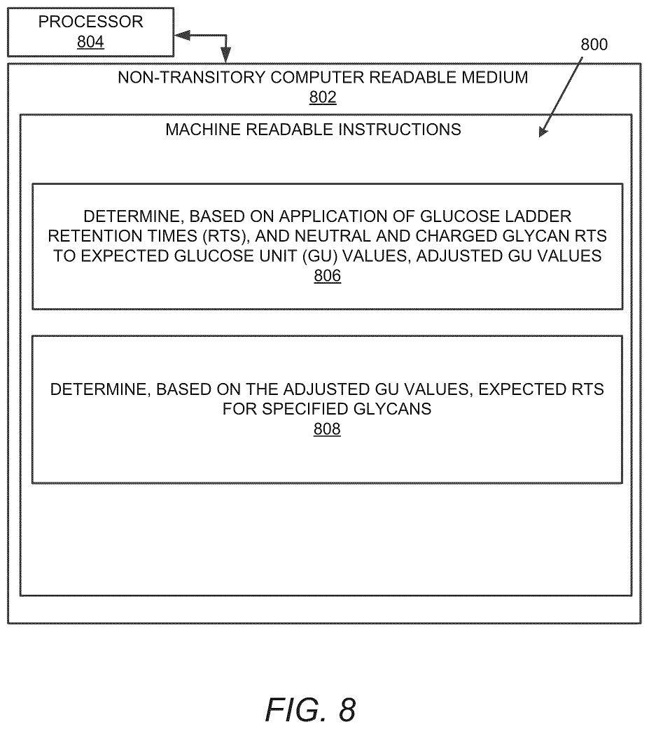

Referring to FIG. 1, the apparatus 100 may include a retention time (RT) receiver 102 that is executed by at least one hardware processor (e.g., the hardware processor 602 of FIG. 6, and/or the hardware processor 804 of FIG. 8) to receive glucose ladder retention times (RTs) 104. Further, the RT receiver 102 may receive neutral and charged glycan RTs 106.

An RT analyzer 108 that is executed by at least one hardware processor (e.g.,. the hardware processor 602 of FIG. 6, and/or the hardware processor 804 of FIG. 8) may determine, based on application of the glucose ladder RTs 104, and the neutral and charged glycan RTs 106 to expected glucose unit (GU) values, adjusted GU values 110.

An expected RT generator 112 that is executed by at least one hardware processor (e.g., the hardware processor 602 of FIG. 6, and/or the hardware processor 804 of FIG. 8) may determine, based on the adjusted GU values 110, expected RTs 114 for specified glycans.

A compound identifier 128 that is executed by at least one hardware processor (e.g., the hardware processor 602 of FIG. 6, and/or the hardware processor 804 of FIG. 8) may identify, based on the expected RTs 114, at least one compound 130.

According to examples disclosed herein, the RT analyzer 108 may determine, based on application of the glucose ladder RTs 104, and the neutral and charged glycan RTs 106 to the expected GU values, the adjusted GU values 110 by determining, based on the glucose ladder RTs 104, a fifth order polynomial GU as a function of RT.

According to examples disclosed herein, the RT analyzer 108 may determine, based on application of the glucose ladder RTs 104, and the neutral and charged glycan RTs 106 to the expected GU values, the adjusted GU values 110 by determining, based on the fifth order polynomial GU and the neutral and charged glycan RTs, an observed GU 116.

According to examples disclosed herein, the RT analyzer 108 may determine, based on application of the glucose ladder RTs 104, and the neutral and charged glycan RTs 106 to the expected GU values, the adjusted GU values 110 by determining, based on the observed GU 116, GU adjustment factors 118. Further, the RT analyzer 108 may determine, based on application of the GU adjustment factors 118 to the expected GU values, the adjusted GU values 110.

According to examples disclosed herein, the GU adjustment factors 118 may include a first value that represents a neutral glycan adjustment value that may be selectable from a set of options that exist in the GU library. In some cases this will allow for the use of a glycan in a sample, for which the identity is known, to perform the adjustment. Since this adjustment is performed with data from the sample run, the accuracy may be further enhanced by eliminating run-to-run variation from this aspect of the calibration. The GU adjustment factors 118 may include a second value that represents an acidic glycan adjustment value applied to S2 glycans that may be similarly selectable from a set of options which exist in the GU library. This will similarly allow for the use of a glycan in a sample, for which the identity is known, to perform the adjustment. The GU adjustment factors 118 may include a third value that represents an acidic glycan adjustment value multiplied by a factor of 0.5 and applied to S1 glycans. The GU adjustment factors 118 may include a fourth value that represents an acidic glycan adjustment value multiplied by a factor of 1.5 and applied to S3 glycans. The GU adjustment factors 118 may include a fifth value that represents an acidic glycan adjustment value multiplied by a factor of 2.0 and applied to S4 glycans.

According to examples disclosed herein, the RT analyzer 108 may determine, based on application of the glucose ladder RTs 104, and the neutral and charged glycan RTs 106 to the expected GU values, the adjusted GU values 110 by determining, based on subtraction of the GU adjustment factors from respective expected GU values, the adjusted GU values.

According to examples disclosed herein, the RT analyzer 108 may determine, based on application of the glucose ladder RTs 104, and the neutral and charged glycan RTs 106 to the expected GU values, the adjusted GU values 110 by determining, based on the glucose ladder RTs, a fifth order polynomial RT as a function of the GU.

According to examples disclosed herein, the expected RTs 114 may be exported, for example, as a .CSV file 120 that contains, in the following order of comma delimited columns: the chemical formulae of each glycan structure (e.g. glycan formulas 122), the expected RTs 114 of each glycan in the library, a blank column, the name of the glycan structure (e.g., glycan names 124), and a final column for comments. This .CSV file 120 may be formatted, for example, by an LC/MS data analysis formatter 126 and loaded by the compound identifier 128 as a compound database file in a data analysis software package, such as MassHunter Data Analysis or Masshunter Bioconfirm. The data analysis software package may be utilized by the compound identifier 128 for automatic searching through LC/MS data for signals that match the expected masses of compounds in a compound database. For example, the .CSV file 120 may be loaded in a data analysis software package as a compound library to be used for automatic analysis of the LC/MS data. The LC/MS data may be generated by LC/MS acquisition software and an LC/MS instrument that is used to analyze a user's sample. Examples of compounds may include InstantPC (IPC) labeled glycan structures G0F, G2F, G2S2, Man5, Man6. Other examples may also include 2AB labeled glycan structures if a user is working with a 2AB labeling sample prep instead of IPC.

The compound identifier 128 may output a table of compounds (e.g., including the compound 130) detected along with a certainty score, and the certainty score may be based on how closely the observed mass for a given compound fits the expected mass based on the chemical formula. The certainty score may range from 0 to 100, where 100 indicates the best possible match between expected and observed values. In this regard the compound identifier 128 may utilize a scalable system where the expected deviation can be edited in the software (e.g., to account for different experimental set ups having different levels of reproducibility), which affects how quickly the score drops off from 100 as the observed value deviates from expected value. Further, expected mass may be determined by the compound identifier 128 from the chemical formula of the compound, and may be independent of RT.

The certainty score may be utilized as an indicator to identify compounds for signals observed in a user's LC/MS data. Thus, by utilizing the certainty score, the compound identifier 128 may identify, based on the expected RTs 114, at least one compound 130.

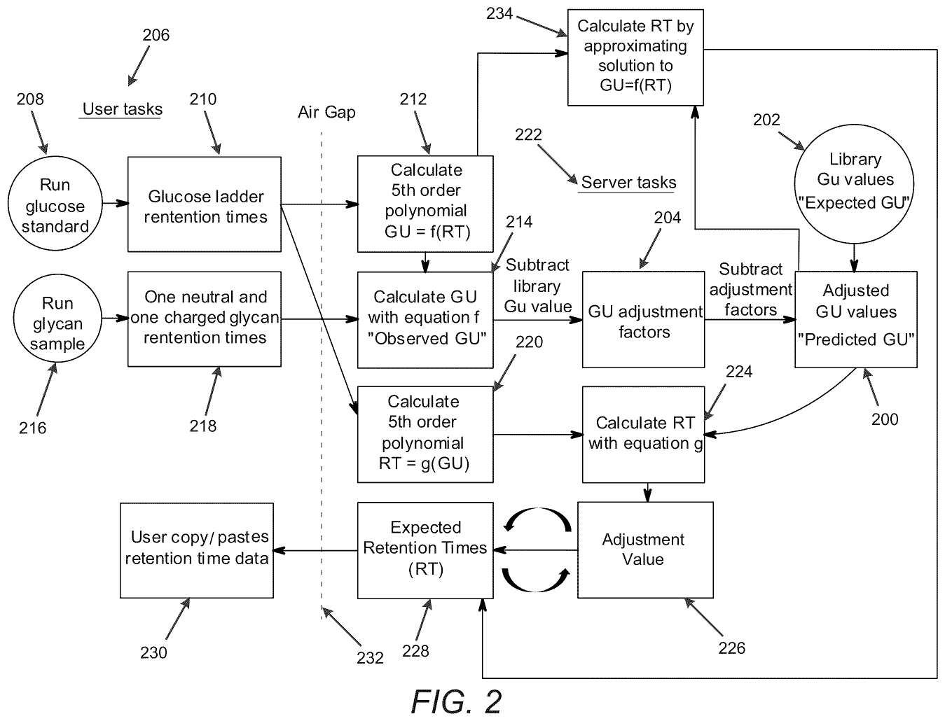

FIG. 2 illustrates a flow chart of utilization and determination of observed glucose units (GU), expected GU, and predicted GU to illustrate operation of the apparatus 100, in accordance with an example of the present disclosure.

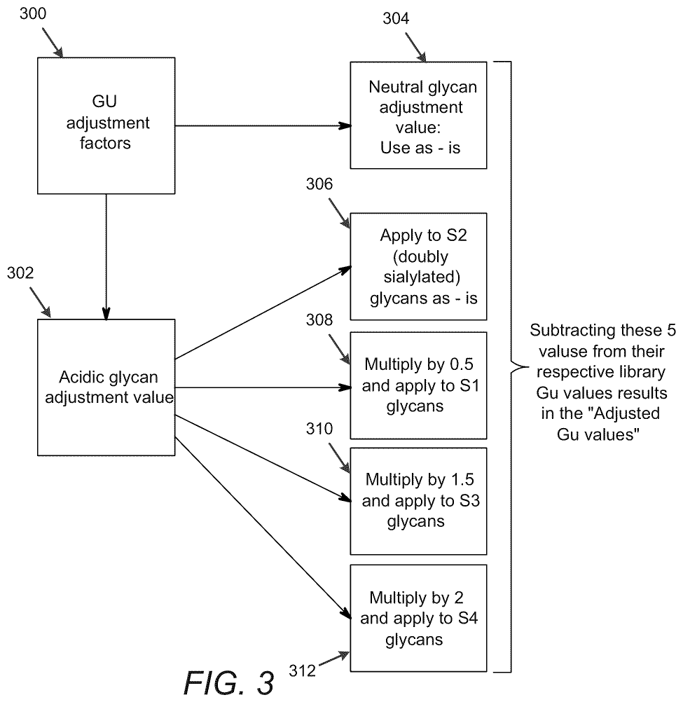

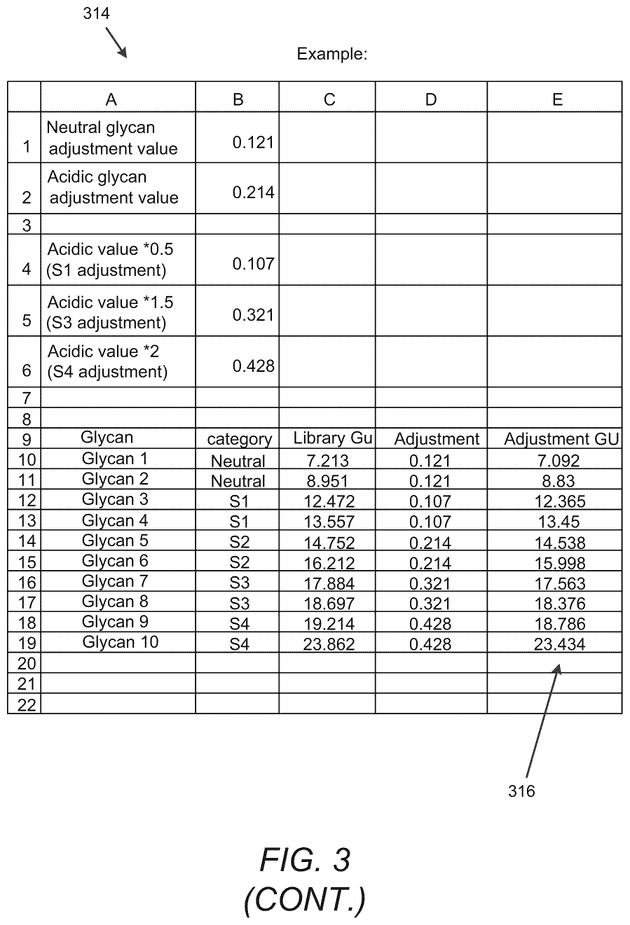

Referring to FIG. 2, at 200, the apparatus 100 may apply (e.g., by the RT analyzer 108) an adjustment to the expected GU (or RT) of each glycan structure in a library of expected GU/RT values at 202 according to the number of acidic monosaccharides it contains, which can be 0, 1, 2, 3 or 4. These “charge groups” of glycans are known, respectively, as S0, S1, S2, S3 and S4. The magnitude of the adjustment applied by the RT analyzer 108 at 204 (further details provided with respect to FIG. 3) to the GU (or expected RT) may be determined using the RT of labeled glycans of known structure, either as adjacent, external RT standards, or internal RT standards if some peaks in the sample itself have been confidently assigned. For example, the observed GU of an S0 glycan such as GOF may be determined and then the deviation from the expected value (from a “library” of expected GU values) may be applied as an adjustment to the predicted GU/RT of all other S0 glycans. In this regard, FIG. 2 illustrates a flow chart of utilization and determination of observed glucose units (GU), expected GU, and predicted GU to illustrate operation of the apparatus 100, in accordance with an example of the present disclosure.

Specifically, referring to FIG. 2, with respect to user tasks at 206, at 208, a user may run a glucose standard, which may be utilized for glucose ladder RTs at block 210 that are further received by the RT analyzer 108 at block 212 to determine a 5^{th} order polynomial GU=f(RT). The output from block 212 may be utilized by the RT analyzer 108 at block 214 to determine GU with equation f (observed GU).

At 216, a user may run a glycan sample, which may be utilized for one neutral and one charged glycan RTs at block 218, which are also received by the RT analyzer 108 at block 214 to determine GU with equation f (observed GU).

The glucose ladder RTs at block 210 may be further received by the RT analyzer 108 at block 220 to determine a 5^{th} order polynomial RT=g(GU).

The operations at blocks 212, 214, and 220, as well as the operations as blocks 200, 202, 204, 224, 226, and 228 may be performed at a server at 222.

The output of blocks 212, 214, and 220 may be utilized by the RT analyzer 108 at block 224 to determine RT with equation g.

The determined RT from block 224 may be utilized with equation f (e.g., block 212), and at block 226, the difference between the result and the predicted GU may be determined. This difference may be divided by the slope (first derivative) of equation f at the x-value RT (calculated at block 224) to generate an adjustment value at block 226, which is then subtracted from the RT of block 224 to determine (e.g., by the expected RT generator 112) expected RTs 114 at block 228. This resulting RT (block 228) may be fed back through the process in block 226 in an iterative fashion, if desired, to generate increasingly accurate expected RTs.

With respect to determination of expected RTs 114 at block 228, referring to block 234, in an alternative approach, a GU value may be entered into equation f to solve for RT (e.g., GU=f(RT)). In this regard, the implementation at block 224 may represent a 1^{st} approximation of the RT, and the adjustment at block 226 may represent a refinement of that approximation. This alternative approach would also eliminate blocks 220, 224, and 226. Moreover, the circular arrows between blocks 226 and 228 show that the adjustment in block 226 can be performed multiple times to obtain better approximations (e.g., the iterative approach as disclosed herein).

Further, at block 230, the expected RTs 114 may be provided to a user.

The apparatus 100 provides an air gap 232 that separates a user's personal data (e.g., at 206) from the software platform (e.g., at 222). A user may likely collect both confidential and non-confidential experimental data, but they are only required to enter non-confidential data into the software platform for the apparatus 100. This data may be processed by the software platform and returned to the user as a list of predicted RTs, which assists the user in interpreting their data.

FIG. 3 illustrates a flow chart of a scaling process to illustrate operation of the apparatus 100, in accordance with an example of the present disclosure.

The adjustment applied by the RT analyzer 108 at block 204 of FIG. 2 is described in further detail in FIG. 3. Referring to FIG. 3, for S2 glycans, the observed deviation of GU/RT of an observed S2 glycan peak of known structure may be determined and applied as an adjustment to the predicted GU/RT of all other S2 glycans. This may be performed for all charge groups, or even other types of structural glycan categories such as high mannose glycans.

However, to limit the burden of obtaining many calibration points, effective adjustment values for S1, S3 and S4 glycans may be determined by scaling up or down the adjustments from the S0 or S2 glycans.

The apparatus 100 thus allows a user to enter observed RTs for their glucose homopolymer standard and enter RTs for an S0 and an S2 glycan. A library of GU values may be provided for labelled glycans that have been determined experimentally using an

HILIC column and separation method. When the apparatus 100 is operated after submittal of calibration RTs by a user, the apparatus may determine the predicted RTs at which the user should expect to see each glycan in a library on their own system.

Referring to FIG. 3, at block 300, GU adjustment factors may be obtained from block 204 of FIG. 2. The GU adjustment factors may be utilized for acidic glycan adjustment values at block 302, and for neutral glycan adjustment value at block 304. The neutral glycan adjustment value at block 304 may represent a first value. The acidic glycan adjustment values at block 302 may be utilized to determine adjusted GU values 110. For example, the acidic glycan adjustment values at block 302 may be used to determine a second value at block 306 based on application to S2 (doubly sialylated) glycans as-is. The acidic glycan adjustment values at block 302 may be used to determine a third value at block 308 based on multiplication by 0.5 and application to S1 glycans. The acidic glycan adjustment values at block 302 may be used to determine a fourth value at block 310 based on multiplication by 1.5 and application to S3 glycans. The acidic glycan adjustment values at block 302 may be used to determine a fifth value at block 312 based on multiplication by 2 and application to S4 glycans. These first to fifth values may be subtracted from their respective library GU values to determine the adjusted GU values 110.

An example of implementation of the flow of FIG. 3 is shown in table 314 of FIG. 3. For example, the neutral glycan adjustment value at block 304 (e.g., first value) is specified as 0.121, the acidic glycan adjustment value from block 302, which is applied to S2 glycans as-is at block 306 (e.g., second value) is specified as 0.214, the third value from block 308 is specified as 0.107, the fourth value from block 310 is specified as 0.321, and the fifth value from block 312 is specified as 0.428. In this regard, as shown at 316, the adjusted GU is shown corresponding to glycans 1-10.

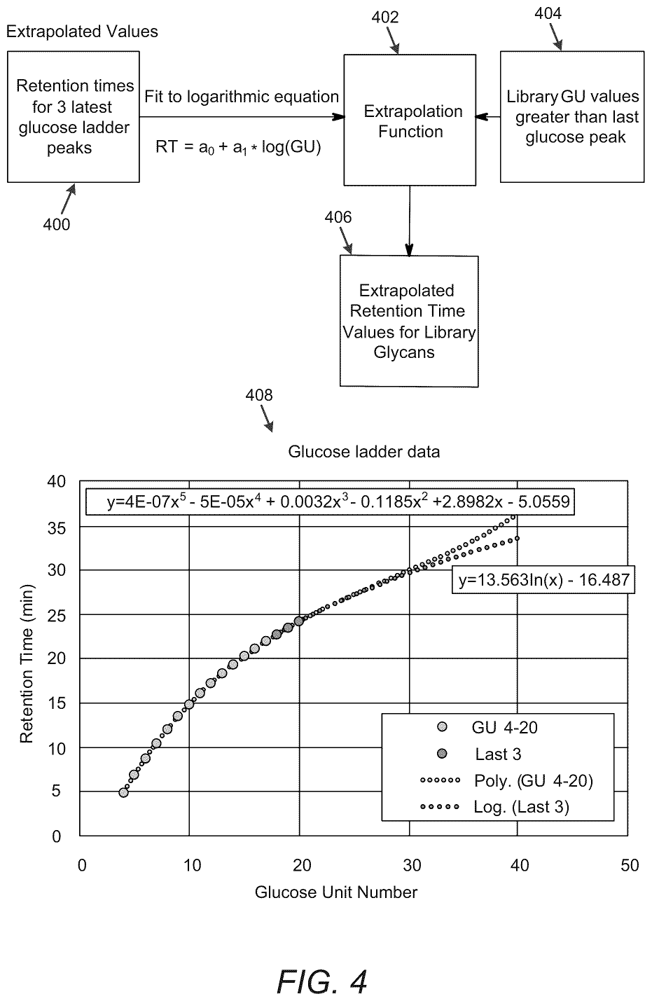

FIG. 4 illustrates a flow chart and an associated example of generation of hypothetical retention times (RTs) for degree of polymerizations beyond those observed experimentally by fitting to a function to illustrate operation of the apparatus 100, in accordance with an example of the present disclosure.

Referring to FIG. 4, for the apparatus 100, as disclosed herein, some glycans elute at an RT that is greater than that of the highest degree of polymerization peaks that can reliably be integrated using available standards of labelled glucose homopolymer. This presents technical challenges when interpolating from a 5^{th} degree polynomial which is valid from the lowest degree of polymerization up until the highest degree of polymerization that was used to fit the function. In this regard, hypothetical RTs may be generated for degree of polymerizations beyond those observed experimentally by fitting to a function, such as a linear function, based on the final 2 or 3 points (e.g., the RTs of the highest, second highest and third highest degree of polymerization labelled glucose homopolymer peaks). These hypothetical glucose homopolymer degree of polymerization retention times may be treated as though they were experimentally observed, allowing the 5^{th} degree polynomial to extend to higher degree of polymerization (and thus higher GU/RT) than would otherwise be possible. In this regard, FIG. 4 illustrates a flow chart and an associated example of generation of hypothetical retention times (RTs) for degree of polymerizations (d.p.s) beyond those observed experimentally by fitting to a function to illustrate operation of the apparatus 100, in accordance with an example of the present disclosure.

Referring to FIG. 4, at block 400, with respect to extrapolated values, RTs for the last three latest glucose ladder peaks may be used to fit to a logarithmic equation (e.g., RT=a0+a1*log(GU)). The results from block 400 may be received by an extrapolation function at block 402. The extrapolation function at block 402 may further receive GU values greater than the last glucose peak from block 404, and generate extrapolated RT values for library glycans at block 406.

An example graph of glucose ladder data is shown at 408. The graph of glucose ladder data shows 5^{th} degree polynomial y=4E-07x⁵−5E-05x⁴+0.0032x³−0.1185x²+2.8982x−5.0559. Further, the graph of glucose ladder data shows a logarithmic equation y=13.563ln(x)−16.487.

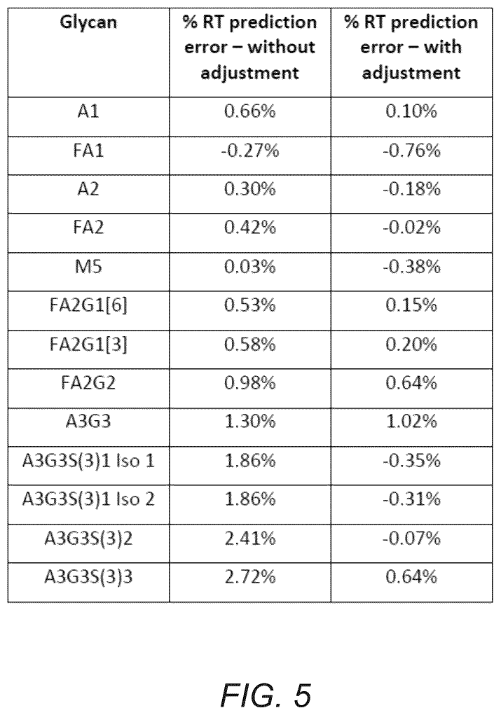

FIG. 5 illustrates improvement in RT prediction accuracy upon implementing an adjustment value to illustrate operation of the apparatus 100, in accordance with an example of the present disclosure.

Referring to FIG. 5, it is shown that upon implementing an adjustment value as disclosed herein, the % RT prediction error decreases, which aids in accurately determining chromatographic peak identities.

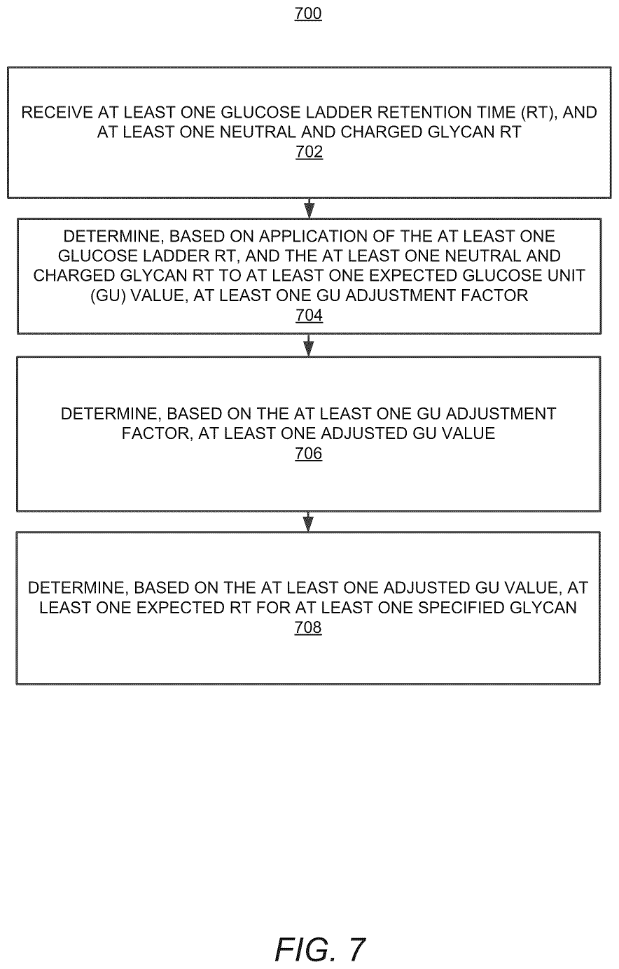

FIGS. 6-8 respectively illustrate an example block diagram 600, a flowchart of an example method 700, and a further example block diagram 800 for glycan peak assignment, according to examples. The block diagram 600, the method 700, and the block diagram 800 may be implemented on the apparatus 100 described above with reference to FIG. 1 by way of example and not of limitation. The block diagram 600, the method 700, and the block diagram 800 may be practiced in other apparatus. In addition to showing the block diagram 600, FIG. 6 shows hardware of the apparatus 100 that may execute the instructions of the block diagram 600. The hardware may include a processor 602, and a memory 604 storing machine readable instructions that when executed by the processor cause the processor to perform the instructions of the block diagram 600. The memory 604 may represent a non-transitory computer readable medium. FIG. 7 may represent an example method for glycan peak assignment, and the steps of the method. FIG. 8 may represent a non-transitory computer readable medium 802 having stored thereon machine readable instructions to provide glycan peak assignment according to an example. The machine readable instructions, when executed, cause a processor 804 to perform the instructions of the block diagram 800 also shown in FIG. 8.

The processor 602 of FIG. 6 and/or the processor 804 of FIG. 8 may include a single or multiple processors or other hardware processing circuit, to execute the methods, functions and other processes described herein. These methods, functions and other processes may be embodied as machine readable instructions stored on a computer readable medium, which may be non-transitory (e.g., the non-transitory computer readable medium 802 of FIG. 8), such as hardware storage devices (e.g., RAM (random access memory), ROM (read only memory), EPROM (erasable, programmable ROM), EEPROM (electrically erasable, programmable ROM), hard drives, and flash memory). The memory 604 may include a RAM, where the machine readable instructions and data for a processor may reside during runtime.

Referring to FIGS. 1-6, and particularly to the block diagram 600 shown in FIG. 6, the memory 604 may include instructions 606 to receive glucose ladder retention times (RTs) 104.

The processor 602 may fetch, decode, and execute the instructions 608 to receive neutral and charged glycan RTs 106.

The processor 602 may fetch, decode, and execute the instructions 610 to determine, based on application of the glucose ladder RTs 104, and the neutral and charged glycan RTs 106 to expected glucose unit (GU) values, adjusted GU values 110.

The processor 602 may fetch, decode, and execute the instructions 612 to determine, based on the adjusted GU values 110, expected RTs 114 for specified glycans.

The processor 602 may fetch, decode, and execute the instructions 614 to identify, based on the expected RTs, at least one compound 130.

Referring to FIGS. 1-5 and 7, and particularly FIG. 7, for the method 700, at block 702, the method may include receiving at least one glucose ladder retention time (RT) 104, and at least one neutral and charged glycan RT 106.

At block 704, the method may include determining, based on application of the at least one glucose ladder RT 104, and the at least one neutral and charged glycan RT 106 to at least one expected glucose unit (GU) value, at least one GU adjustment factor 118.

At block 706, the method may include determining, based on the at least one GU adjustment factor 118, at least one adjusted GU value 110.

At block 708, the method may include determining, based on the at least one adjusted GU value 110, at least one expected RT 114 for at least one specified glycan.

Referring to FIGS. 1-5 and 8, and particularly FIG. 8, for the block diagram 800, the non-transitory computer readable medium 802 may include instructions 806 to determine, based on application of glucose ladder retention times (RTs) 104 to expected glucose unit (GU) values, and neutral and charged glycan RTs 106, adjusted GU values 110.

The processor 804 may fetch, decode, and execute the instructions 808 to determine, based on the adjusted GU values 110, expected RTs 114 for specified glycans.

What has been described and illustrated herein is an example along with some of its variations. The terms, descriptions and figures used herein are set forth by way of illustration only and are not meant as limitations. Many variations are possible within the spirit and scope of the subject matter, which is intended to be defined by the following claims—and their equivalents—in which all terms are meant in their broadest reasonable sense unless otherwise indicated.

What is claimed is:

1. A glycan peak assignment apparatus comprising:
a retention time (RT) receiver, executed by at least one hardware processor, to:
receive glucose ladder retention times (RTs); and
receive neutral and charged glycan RTs;
a RT analyzer, executed by the at least one hardware processor, to:
determine, based on application of the glucose ladder RTs, and the neutral and charged glycan RTs to expected glucose unit (GU) values, adjusted GU values;
an expected RT generator, executed by the at least one hardware processor, to:
determine, based on the adjusted GU values, expected RTs for specified glycans; and
a compound identifier, executed by the at least one hardware processor, to:
identify, based on the expected RTs, at least one compound.

2. The glycan peak assignment apparatus according to claim 1, wherein the RT analyzer is executed by the at least one hardware processor to determine, based on application of the glucose ladder RTs, and the neutral and charged glycan RTs to the expected GU values, the adjusted GU values by:
determining, based on the glucose ladder RTs, a fifth order polynomial GU as a function of RT.

3. The glycan peak assignment apparatus according to claim 2, wherein the RT analyzer is executed by the at least one hardware processor to determine, based on application of the glucose ladder RTs, and the neutral and charged glycan RTs to the expected GU values, the adjusted GU values by:
determining, based on the fifth order polynomial GU and the neutral and charged glycan RTs, an observed GU.

4. The glycan peak assignment apparatus according to claim 3, wherein the RT analyzer is executed by the at least one hardware processor to determine, based on application of the glucose ladder RTs, and the neutral and charged glycan RTs to the expected GU values, the adjusted GU values by:
determining, based on the observed GU, GU adjustment factors; and
determining, based on application of the GU adjustment factors to the expected GU values, the adjusted GU values.

5. The glycan peak assignment apparatus according to claim 4, wherein the GU adjustment factors include a first value that represents a neutral glycan adjustment value.

6. The glycan peak assignment apparatus according to claim 4, wherein the GU adjustment factors include a second value that represents an acidic glycan adjustment value applied to S2 glycans.

7. The glycan peak assignment apparatus according to claim 4, wherein the GU adjustment factors include a third value that represents an acidic glycan adjustment value multiplied by a factor of 0.5 and applied to S1 glycans.

8. The glycan peak assignment apparatus according to claim 4, wherein the GU adjustment factors include a fourth value that represents an acidic glycan adjustment value multiplied by a factor of 1.5 and applied to S3 glycans.

9. The glycan peak assignment apparatus according to claim 4, wherein the GU adjustment factors include a fifth value that represents an acidic glycan adjustment value multiplied by a factor of 2.0 and applied to S4 glycans.

10. The glycan peak assignment apparatus according to claim 4, wherein the RT analyzer is executed by the at least one hardware processor to determine, based on application of the glucose ladder RTs, and the neutral and charged glycan RTs to the expected GU values, the adjusted GU values by:
determining, based on subtraction of the GU adjustment factors from respective expected GU values, the adjusted GU values.

11. The glycan peak assignment apparatus according to claim 1, wherein the RT analyzer is executed by the at least one hardware processor to determine, based on application of the glucose ladder RTs, and the neutral and charged glycan RTs to the expected GU values, the adjusted GU values by:
determining, based on the glucose ladder RTs, a fifth order polynomial RT as a function of GU.

12. A method for glycan peak assignment, the method comprising:
receiving, by at least one hardware processor, at least one glucose ladder retention time (RT), and at least one neutral and charged glycan RT;
determining, by the at least one hardware processor, based on application of the at least one glucose ladder RT, and the at least one neutral and charged glycan RT to at least one expected glucose unit (GU) value, at least one GU adjustment factor; and
determining, by the at least one hardware processor, based on the at least one GU adjustment factor, at least one adjusted GU value; and
determining, by the at least one hardware processor, based on the at least one adjusted GU value, at least one expected RT for at least one specified glycan.

13. The method according to claim 12, wherein determining, by the at least one hardware processor, based on the at least one GU adjustment factor, the at least one adjusted GU value further comprises:
determining, based on subtraction of the at least one GU adjustment factor from at least one expected GU value, the at least one adjusted GU value.

14. A non-transitory computer readable medium having stored thereon machine readable instructions, the machine readable instructions, when executed by at least one hardware processor, cause the at least one hardware processor to:
determine, based on application of glucose ladder retention times (RTs), and neutral and charged glycan RTs to expected glucose unit (GU) values, adjusted GU values;
determine, based on the adjusted GU values, expected RTs for specified glycans.

15. The non-transitory computer readable medium according to claim 14, wherein the machine readable instructions to determine, based on application of the glucose ladder RTs, and the neutral and charged glycan RTs to expected GU values, the adjusted GU values, when executed by the at least one hardware processor, further cause the at least one hardware processor to:
determine, based on the glucose ladder RTs, a fifth order polynomial GU as a function of RT.

16. The non-transitory computer readable medium according to claim 15, wherein the machine readable instructions to determine, based on application of the glucose ladder RTs, and the neutral and charged glycan RTs to expected GU values, the adjusted GU values, when executed by the at least one hardware processor, further cause the at least one hardware processor to:
determine, based on the fifth order polynomial GU and the neutral and charged glycan RTs, an observed GU.

17. The non-transitory computer readable medium according to claim 16, wherein the machine readable instructions to determine, based on application of the glucose ladder RTs, and the neutral and charged glycan RTs to expected GU values, the adjusted GU values, when executed by the at least one hardware processor, further cause the at least one hardware processor to:
determine, based on the observed GU, GU adjustment factors; and
determine, based on application of the GU adjustment factors to the expected GU values, the adjusted GU values.

18. The non-transitory computer readable medium according to claim 17, wherein the GU adjustment factors include:
a first value that represents a neutral glycan adjustment value;
a second value that represents an acidic glycan adjustment value applied to S2 glycans;
a third value that represents the acidic glycan adjustment value multiplied by a factor of 0.5 and applied to S1 glycans;
a fourth value that represents the acidic glycan adjustment value multiplied by a factor of 1.5 and applied to S3 glycans; and
a fifth value that represents the acidic glycan adjustment value multiplied by a factor of 2.0 and applied to S4 glycans.

19. The non-transitory computer readable medium according to claim 17, wherein the machine readable instructions to determine, based on application of the glucose ladder RTs, and the neutral and charged glycan RTs to expected GU values, the adjusted GU values, when executed by the at least one hardware processor, further cause the at least one hardware processor to:
determine, based on subtraction of the GU adjustment factors from respective expected GU values, the adjusted GU values.

20. The non-transitory computer readable medium according to claim 14, wherein the machine readable instructions, when executed by the at least one hardware processor, further cause the at least one hardware processor to:
generate, based on the expected RTs for the specified glycans, a library of the expected RTs; and
assign, based on the expected RTs from the library of the expected RTs, signals observed in liquid chromatography (LC)/mass spectrometry (MS) data to glycan structures.
